(19) United States
(12) Patent Application Publication
(10) Pub. No.: US 2009/0181090 A1
(43) Pub. Date: Jul. 16, 2009

(54) Protein-Based Carrier System for Overcoming Resistance in Tumour Cells

(76) Inventors: Sebastian Dreis, Frankfurt (DE); Klaus Langer, Schöneck (DE); Jörg Kreuter, Bad Homburg (DE); Martin Michaelis, Frankfurt (DE); Jindrich Cinatl, Offenbach (DE)

Correspondence Address: D. PETER HOCHBERG CO. L.P.A., 1940 EAST 6TH STREET, CLEVELAND, OH, 44114, US

(21) Appl. No.: 12/087,175

(22) PCT Filed: Dec. 22, 2006
(86) PCT No.: PCT/EP2006/012524

§ 371 Date: Jun. 27, 2008

(30) Foreign Application Priority Data
Dec. 27, 2005 (DE) 10 2005 062 440.5

(51) Int. Cl. A61K 9/14; A61K 47/42; A61K 31/704; A61K 39/395; A61P 35/00

(52) U.S. Cl. 424/484; 514/773; 514/34; 424/141.1; 514/776; 514/774; 514/775; 977/773; 977/906

(57) ABSTRACT

Nanoparticles, the particle matrix of which is based on at least one protein and has at least one active agent embedded therein and methods of producing the nanoparticles with at least one active agent embedded in the protein matrix are disclosed. The use of the nanoparticles for the treatment of tumours, in particular for the treatment of tumours which are resistant to chemical medicaments is also disclosed.

### CROSS-REFERENCE TO RELATED APPLICATIONS

This application is a National Stage application of International Application No. PCT/EP2006/012524, filed on Dec. 22, 2006, which claims priority of German application number 10 2005 062 440.5, filed on Dec. 27, 2005, both of which are incorporated herein by reference in their entireties.

### BACKGROUND OF THE INVENTION

1. Field of the Invention

The present invention relates to treating tumours which are resistant to chemotherapeutic agents. More particularly, the present invention relates to the use of nanoparticles comprising a matrix for manufacturing a medicament for treating tumours which are resistant to chemotherapeutic agents.

2. Description of the Prior Art

The development of resistance in the treatment of solid tumours poses a great problem in oncology. Resistance is frequently due to increased excretion of the chemotherapeutic substances by the tumour cells. The mechanism of this resistance development is linked to the overexpression of P-glycoprotein (Pgp) [Krishna et al., (2000), Eur. J. Pharm. Sci. 11, 265]. Pgp is an ATP-dependent efflux pump, which is able to actively extrude medicinal substances from tumour cells. The overexpression of Pgp results in a decreased accumulation of the chemotherapeutic agent in the cells, so that its intracellular concentration does not suffice for achieving an antineoplastic effect. To compensate for the reduced accumulation of the chemotherapeutic agent, a dose adaptation, i.e. a dose increase, of the cytostatic agent is required, which, however, is limited because of the toxic side effects of the cytostatic which accompany such an increase. The overexpression of Pgp leads to so-called multiresistance [multidrug resistance, MDR), where the cell is resistant not only to the original substance but, in addition, to a plurality of cytostatics. This phenomenon considerably limits the success of tumour chemotherapy.

In the past, various approaches have been developed to tumour cell resistance. The most frequently examined approach is the use of active agents which act as inhibitors of Pgp. It was already in 1981 that the inhibitory effect of calcium antagonists on Pgp was established [Tsuruo et al., (1981), Cancer Res. 41, 1967]. In these studies, an increased accumulation of vincristine and doxorubicin in vincristine-resistant P388 tumour cells was observed when these tumour cells were additionally incubated with a calcium antagonist. A promising member of the active agent group of the calcium antagonists turned out to be verapamil. But other active agents, such as cyclosporin A, too, are potent inhibitors of Pgp, as could be shown [Slater et al., (1986), J. Clin. Invest. 77, 1405]. In these studies, the resistance of acute lymphatic leukaemia cells to vincristine and daunorubicin could be overcome by simultaneous administration of cyclosporin A.

Since both verapamil and cyclosporin A have many potential side effects, further Pgp inhibitors were sought. Thus, the multiresistance of the P388/ADM and K562/ADM cells was overcome in in-vitro experiments using the two Pgp inhibitors MS-209 and SDZ PSC 833 [Naito et al., (1997), Cancer Chemother. Pharmacol. 40, Suppl. S20].

Another strategy for overcoming multiresistance is the chemical modification of active agents. This strategy attempts to overcome the resistance of tumour cells by conjugating antineoplastic active agents with different macromolecules. The macromolecules here serve as carriers for the active agent. This is also called a carrier system.

Already in 1992 it was shown that the Pgp-mediated resistance in various cancer cell lines can be overcome with doxorubicin-loaded polyisohexylcyanoacrylate (PIHCA) nanospheres [Cuvier et al., (1992), Biochem. Pharmacol. 44, 509]. These trials were confirmed with doxorubicin-resistant C6 cells wherein the inhibitory concentration 50 (IC50) of doxorubicin-loaded polyisohexylcyanoacrylate nanospheres was significantly lower than that of unconjugated doxorubicin [Bennis et al., (1994), Eur. J. Cancer 30A, 89]. This result could also be confirmed on hepatocellular carcinoma cells, using corresponding doxorubicin-loaded PIHCA nanoparticles [Barraud et al., (2005), J. Hepatol. 42, 736].

The mechanism of overcoming resistance by colloidal carrier systems initially gave rise to speculations. According to one wide-spread opinion, such carrier systems were taken up by the target cells via an endocytotic process, thus bypassing the Pgp-mediated resistance mechanisms. In relation to polyisohexylcyanoacrylate nanoparticles, this opinion was proved wrong [Henry-Toulme et al., (1995), Biochem. Pharmacol. 50, 1135]. In fluorescence microscopic studies of resistant tumour cell lines after incubation with PIHCA nanoparticles, no accumulation of particles was observed in the cells, whereas accumulation in phagocyting cells, such as macrophages, could be shown. Overcoming multiresistance by PIHCA nanoparticles was therefore discussed as being a synergism of products of the polymer matrix and the active agent. This hypothesis is supported by examinations which showed that doxorubicin-loaded polyisobutylcyanoacrylate (PIBCA) nanoparticles had an increased cytotoxic effect on resistant P388/Adr cells [Colin de Verdiere et al., (1994), Cancer Chemother. Pharmacol. 33, 504]. Incubation of the cells with PIBCA nanoparticles led to a five-fold increase of the active agent concentration in the target cells. A nanoparticle/cell interaction was discussed as being the mechanism at the base of this phenomenon, in contrast to an endocytotic uptake of the nanoparticles.

In 1993, Ohkawa et al. published a study on the effect of doxorubicin bovine serum albumin conjugates on resistant rat hepatoma cells (AH66DR) [Cancer Res. 53, 4238-4242]. The doxorubicin-bovine serum conjugates showed an increased cytotoxic effect compared to the control with unmodified active agent. An increased accumulation of the conjugates due to a reduced efflux was discussed as being the cause of this effect. The treatment of peritoneal tumour-bearing rats showed that the doxorubicin bovine serum albumin conjugates increased the mean survival rate from 30 days in the control group to 50 days.

The doxorubicin bovine serum albumin conjugates described by Ohkawa et al. were produced by dissolving the active agent and the bovine serum albumin in a suitable solvent and then adding glutaraldehyde. The glutaraldehyde reacts with functional groups of the active agent and of the target protein, in this case amino groups, and thus leads to a covalent coupling of the molecules. The transport capacity of the doxorubicin bovine serum albumin conjugates is indicated as amounting to three to four active agent molecules per carrier unit.

The doxorubicin bovine serum albumin conjugates described by Ohkawa et al. are thus covalent chemical bonds of doxorubicin to bovine serum albumin. Such a chemical modification of the active agent alters the physicochemical properties of the agent. New active agents are formed (NCI: new chemical entities) that have different and new effects in biological systems.

For the doxorubicin bovine serum albumin conjugates to have an antineoplastic effect it has to be possible to cleave the covalent active agent-protein bond in the target tissue. Only thereby is a release of the therapeutically active agent achieved.

Despite these disadvantages, the use of colloidal “drug delivery systems” or active agent-conjugated carrier systems, such as nanoparticles or nanospheres, is among the promising strategies for overcoming tumour cells.

### SUMMARY OF THE PRESENT INVENTION

It was thus the object of the present invention to provide a colloidal “drug delivery system” for overcoming resistance in tumour cells which does not have the disadvantages of the known conjugates of active agents covalently bound to a carrier material.

This object is solved by providing nanoparticles wherein at least one active agent is enclosed in a matrix of protein but is not covalently coupled to said protein.

The subject matter of the present invention are nanoparticles, the particle matrix of which is based on at least one protein and has at least one active agent embedded therein, methods of production of such nanoparticles, and the use of such nanoparticles for the treatment of tumours and for the manufacture of medicaments for the treatment of tumours, in particular for the treatment of tumours which are resistant to chemical medicaments.

The nanoparticles according to the invention comprise at least one protein, on which the particle matrix is based, and at least one active agent, which is embedded in said matrix.

In principle, any physiologically tolerable, pharmacologically acceptable proteins which are soluble in an aqueous medium are suitable as the protein or proteins forming the matrix of the nanoparticles. Especially preferred proteins are gelatine and albumin, which may originate from different animal species (cattle, pigs etc.), as well as the milk protein casein. In principle, it is also possible to use other proteins as the starting material for producing the nanoparticles according to the invention, for example immunoglobulins.

Basically, any desired active agent with intracellular action can be embedded into the particle matrix. Preferably, however, cytostatics and/or other antineoplastic active agents are to be administered, with the aid of the nanoparticles according to the invention for treating tumours, especially tumours which are resistant to cytostatic drugs or other antineoplastic active agents. Especially preferred nanoparticles have anthracyclines, such as doxorubicin, daunorubicin, epirubicin or idarubicin, embedded in their protein matrix.

Suitable as the antineoplastic agents that may be embedded in the protein matrix of the nanoparticles are, for example:
cytostatic agents,
   plant cytostatic agents, e.g. mistletoe preparations,
   chemically defined cytostatics,
      alkaloids and podophyllotoxins,
         vinca alkaloids and analogues, e.g. vinblastine, vincristine, vindesine, vinorelbine,
      podophyllotoxin derivatives, e.g. etoposide, teniposide,
      alkylating agents,
         nitrosoureas, e.g. nimustine, carmustine, lomustine,
         nitrogen mustard analogues, e. g. cyclophosphamide, estramustine, melphalan, ifosfamide, trofosfamide, chlorambucil, bendamustine,
         other alkylating agents, e. g. dacarbazine, busulfan, procarbazine, treosulfan, temozolomide, thiotepa,
      cytotoxic antibiotics,
         anthracycline-related substances, e. g. mitoxantrone,
         other cytotoxic antibiotics, e. g. bleomycin, mitomycin, Dactinomycin,
      antimetabolites,
         folic acid analogues, e. g. methotrexate
         purine analogues, e. g. fludarabine, cladribine, mercaptopurine, thioguanine
         pyrimidine analogues, e. g. cytarabine, gemcitabine, fluorouracil, capecitabine,
   other cytostatics, e. g. paclitaxel, docetaxel
other antineoplastic agents,
   platinum compounds, e. g. carboplatin, cisplatin, oxaliplatin,
   other antineoplastic agents such as amsacrine, irinotecan, hydroxycarbamide, pentostatin, porfimer sodium, aldesleukin, tretinoin und asparaginase.

It is possible to embed any of the active agents listed in the above list of active agents in the particle matrix of the protein-based carrier system. Because of the different physicochemical properties of the active agents (e.g. solubility, adsorption isotherms, plasma protein bond, pKa values) it may, however, be necessary to optimise the method of production of the active agent-containing nanoparticles for the respective active agent.

The nanoparticles according to the invention thus constitute a protein-based carrier system with at least one active agent which is embedded in the protein matrix of the particles, preferably for the treatment of tumours, particularly for the treatment of resistant tumours.

The nanoparticles according to the invention preferably have a size of 100 to 600 nm, more preferably of 100 to 400 nm. In an especially preferred embodiment, the nanoparticles have a size of I 00 to 200 nm.

The nanoparticles according to the invention are capable of overcoming the resistance of the tumour cells to chemical medicaments.

### BRIEF DESCRIPTION OF THE DRAWINGS

FIG. 1 is a diagram illustrating the influence of doxorubicin nanoparticles (Dxr-NP), doxorubicin solution (Dxr-Soln) and doxorubicin liposomes (Dxr-Lip) on the cell viability of parenteral neuroblastoma cells.

FIG. 2 is a diagram illustrating the influence of doxorubicin nanoparticles (Dxr-NP), doxorubicin solution (Dxr-Soln) and doxorubicin liposomes (Dxr-Lip) on the cell viability of resistant neuroblastoma cells.

### DETAILED DESCRIPTION OF THE PRESENT INVENTION

The nanoparticles according to the present invention may have a modified surface. The surface may, for example, be PEGylated, i.e. polyethylene glycols may be bound to the surface of the nanoparticles by means of covalent bonds. By modifying the surface with polyethylene glycols (PEGs), the properties of the nanoparticles can be altered such that their stability, half-life in the organism, water-solubility, immunological properties and/or bioavailability can be improved.

The nanoparticles may, however, also have “drug targeting ligands” on their surface which enable a targeted accumulation of the nanoparticles in a particular organ or in particular cells. Preferred drug targeting ligands are ligands which recognise tumour-specific proteins. The ligands may be selected, for instance, from the group comprising tumour-specific protein-recognising antibodies, such as trastuzumab and cetuximab, and transferrin as well as galactose. The drug targeting ligands may also be coupled to the surface of the nanoparticles via bifunctional PEG derivatives.

In connection with the modification of the surface of the nanoparticles according to the invention, reference is made herein to WO 2005/089797 A2, the content of which is in its entirety incorporated by reference in the disclosure of the present invention.

Preferably, the nanoparticles according to the invention are produced initially by co-dissolving the active agent/active agents and the protein/the proteins, preferably in water or in an aqueous medium. Subsequently, the protein is precipitated from the solution in a slow and controlled manner by simple desolvation through controlled addition of a non-solvent for the protein, preferably an organic solvent, more preferably ethanol. In the process, the colloidal carrier system (nanoparticles) is formed around the active substance molecules in solution. The active agent is thereby embedded in the matrix of the carrier system without being modified.

When producing the active agent-loaded nanoparticles, the active agent is preferably used in a molar excess, relative to the protein. With particular preference, the molar ratio of active agent to protein is 5:1 up to 50:1. Loading of the nanoparticles in molar ratios of more than 50:1 is also possible.

By subsequent crosslinking of the protein matrix by adding a crosslinking agent, preferably glutaraldehyde, the matrix of the nanoparticles is stabilised.

By varying the amount of crosslinking agent, it is possible to achieve different degrees of stabilisation of the particle matrix. Preferably such nanoparticles are produced which are 50% to 200% stabilised. These percentages relate to the molar ratios of the amino groups present on the protein used to the aldehyde functions of the glutaraldehyde. A molar ratio of 1:1 corresponds to a 100% stabilisation.

Apart from the bifunctional aldehyde glutaraldehyde, other bifunctional substances that are able to form covalent bonds with the protein are suitable for the stabilisation of the protein matrix. These substances can react, for example, with amino groups or sulfhydryl groups of the proteins. Examples for suitable crosslinking agents are formaldehyde, bifunctional succinimides, isothiocyanates, sulfonyl chlorides, maleimides and pyridyl sulphides.

However, a stabilisation of the protein matrix may also be effected by action of heat. Preferably, the protein matrix is stabilised by a two-hour incubation at 70° C. or a one-hour incubation at 80° C.

Because the crosslinking takes place only after the precipitation of the nanoparticles, the carrier system according to the invention does not constitute a chemically covalent bond of an active agent to the protein. Rather, the active substance is embedded in the matrix of the carrier system. Consequently, the integration of the active substance is largely independent from the type of active agent and can be employed universally.

By contrast to covalently bonded active agent conjugates, which necessitate that the active agent-protein bond in the target tissue can be cleaved in order to achieve the release of the active agent, the active agent release in the inventive colloidal carrier system takes place via the degradation of the protein structure by lysosomal enzymes, which are present in all tissues. To this end, a direct cleavage of the active agent-protein bond is not necessary.

The present particle system for overcoming resistance in tumour cells has the following advantages:
1. Overcoming the resistance in tumour cells.
2. Increased cytotoxicity to tumour cells, compared to liposomal preparations and compared to the solution of an active agent.
3. The protein-based nanoparticles consist of a physiological material.
4. Additional medication with Pgp inhibitors is not necessary.
5. The active agent, being located inside the particle matrix, is protected against outside influences.
6. Modification of the particle surfaces is easily possible.

By chemical conversion of the functional groups present on the particle surface (amino groups, carboxyl groups, hydroxyl groups) with suitable chemical reagents, it is possible to bind, for example, polyethylene glycol chains (PEG) of different chain length to the nanoparticles. In this method, which is called PEGylation or protein pegylation, the surface modification of the nanoparticles is essentially brought about by stable, covalent bonds between one amino group or sulfhydryl group on the protein and one chemically reactive group (carbonate, ester, aldehyde or tresylate) on the PEG. The resulting structures may be linear or branched. The PEGylation reaction is influenced by factors such as the mass of the PEGs, the type of protein, the concentration of the protein in the reaction mixture, the reactive time, the temperature and the pH value. Hence, the appropriate PEGs must be found for each carrier system.

Apart from the PEGylation of the particle surface in the narrower sense, i.e. conversion of the protein particles with monofunctional PEG derivatives, it is also possible to bind bifunctional PEG derivatives to the particle surface, in order to couple so-called “drug targeting ligands” to the particles. Other surface modifications are, for example, the conversion of functional groups on the particle surface with acetic acid anhydride or iodoacetic acetic acid in order to attach acetyl groups or acetic acid groups.

The surface of the nanoparticles according to the present invention can also be modified by protein-chemical reactions with an appropriate drug targeting ligand, whereby it is possible to accumulate the nanoparticles in certain organs or cells without having to adapt the carrier system prior thereto.

Any tumour-specific proteins can be used as the receptors for the “drug targeting ligands”. With particular preference, antibodies which recognise tumour-specific proteins, for example the antibodies trastuzumab and cetuximab, are used as the “drug targeting ligands”. Trastuzumab (HERCEPTIN®) recognises HER2 receptors, which are overexpressed by many tumour cells, and is approved for the treatment of breast cancer. Cetuximab (ERBITUX®) recognises the receptor for the epidermal growth factor on a multiplicity of tumour cells and is approved for the treatment of colorectal carcinoma. Apart from antibodies, “drug targeting” can also be achieved via ligands bound to the particles, such as transferrin, which recognises the transferrin receptor which is overexpressed by tumour cells, or via low-molecular receptor ligands such as galactose, which is bound by the asialoglycoprotein receptor on hepatocytes.

### Example of an Embodiment

To produce nanoparticles according to the invention, 20.0 mg human serum albumin and 1.0 mg doxorubicin hydrochloride were dissolved in 1.0 ml of ultrapure water, which corresponds to a molar ratio of 5 to 1 (active agent to protein), and incubated for 2 hours while stirring. When adding 3.0 ml ethanol 96% via a pump system (1.0 ml/min), precipitation of the serum albumin occurred in the form of nanoparticles. These were crosslinked for 24 hours to different extent by addition of different amounts of 8% glutaraldehyde (Table 1). The stabilised nanoparticles were divided into aliquots of 2.0 ml and purified by 3 cycles of centrifugation and redispersion in the ultrasound bath. The supernatants of the individual wash steps were collected and the portion of the un-bound doxorubicin contained therein was determined by RP18 HPLC. To determine the nanoparticle concentration, 50.0 μl of the preparation were applied to a weighed metal boat and dried at 80° C. for 2 hours. After cooling down, the preparation was weighed again and the nanoparticle concentration was calculated.

The efficiency of the loading with doxorubicin was determined by quantification of the unbound portion by RP18-HPLC. The absolute loading, depending on the degree of crosslinking, was 35.0-48.0 μg of active agent per mg of the carrier system. Hence, the transport capacity of the carrier system is about 10⁶ active substance molecules per carrier unit (=nanoparticle).

TABLE 1
Stabilisation of doxorubicin-containing nanoparticles
on the basis of human serum albumin

| | Amount of Glutaraldehyde |
|---|---|
| Stabilisation | (8% solution) |
| 200% | 23.50 μl |
| 100% | 11.75 μl |
| 75% | 5.88 μl |
| 50% | 2.94 μl |

To test the cytotoxicity of the doxorubicin nanoparticles (Dxr-NP) produced, as compared to a doxorubicin solution (Dxr-Soln) and a liposomal doxorubicin preparation (CAELYX®), the following cell lines were used:

parenteral cells of a human neuroblastoma cell line of the Universitätsklinikum Frankfurt (university clinical centre of Frankfurt) (UKF-NB3 Par.)

doxorubicin-resistant cells of the human neuroblastoma cell line of the Universitätsklinikum Frankfurt (UKF-NB3 Dxr-R.)

To determine the cytotoxicity, the MTT test was used. In this test the viability of the cells in the presence of different concentrations of a substance is determined and is then compared with a cell control. From the results, the IC50 value (inhibitory concentration 50), i.e. the concentration of a substance at which 50% of the cells die, can be calculated. This test is based on the reduction of 3-(4,5-dimethyl-2-thiazolyl)-2,5-diphenyl-2H-tetrazolium bromide in the mitochondria of vital cells. By this reduction, the yellow tetrazolium salt is reduced to formazan, which precipitates as blue crystals. After dissolving the crystals with SDS/DMF solution, the colour intensity can be measured photometrically. A high absorption here means high cell viability.

For testing the cytotoxicity in parenteral and resistant neuroblastoma cells, the cells were evenly partitioned into the wells of a 96-well microtitre plate.

One column of the wells contained pure medium and corresponded to the blank value; in a second column the cells for the growth control (100% value) were cultivated. The doxorubicin-comprising preparations (Dxr-NP, Dxr-Soln and Dxr-Lip) were pipetted into the remaining wells, with concentrations increasing from right to left (0.75; 1.5; 3.0; 6.0; 12.5; 25.0; 50.0; 100.0 ng/ml). The microtitre plate was subsequently incubated in the incubator for 5 days at 37° C., with 5% CO₂. 25 μl of MTT solution was pipetted into each well and incubated for 4 hours, again at 37° C. in the incubator. The reduction of the tetrazolium bromide into the blue formazan crystals was stopped by addition of 100 μl SDS/DMF-solution. After a further incubation at 37° C. overnight, the colour crystals had dissolved completely, and the colour intensity in each well was measured photometrically at 620/690 μm. By subtracting the blank value from the measured values and with reference to the control, the cell viability can be expressed in percent.

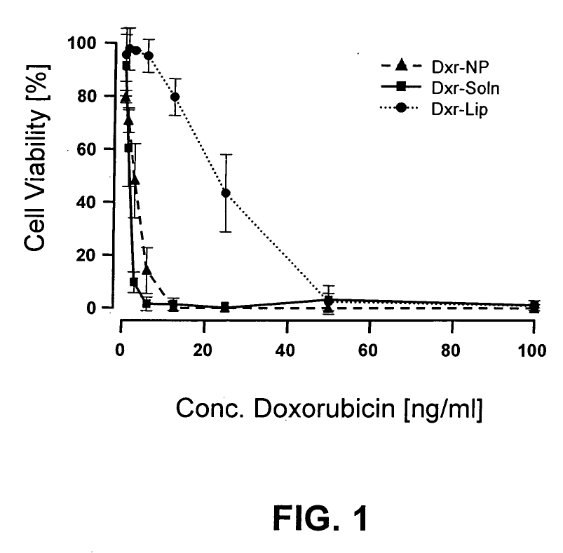

The cytotoxicity of different doxorubicin-containing preparations was tested on a parenteral neuroblastoma cell line (UKF-NB3 Par.) without resistance mechanisms, and on a doxorubicin-resistant neuroblastoma cell line (UKF-NB3 Dxr-R.). Testing of the parenteral cell line (FIG. 1 ) showed that both the Dxr solution and the Dxr-NP with a 100% stabilisation, have a strong cytotoxic effect on parenteral neuroblastoma cells. Already at a low concentration of 3 ng/ml of doxorubicin, the cell viability sank to below 50%. The liposomal Dxr preparation (CAELYX®) showed a markedly lower cytotoxic effect on the cells. With this preparation, higher concentrations of the medicinal substance were required (25.0 ng/ml). This result is confirmed by the calculation of the IC50 value for the individual preparations (Table 2). Dxr-NP and Dxr-Soln caused the death of 50% of the cells already at concentrations of 2.4 ng/ml and 1.6 ng/ml, respectively, whereas the Dxr liposomes, having an IC50 of 25.8 ng/ml, had to be used in considerably higher doses.

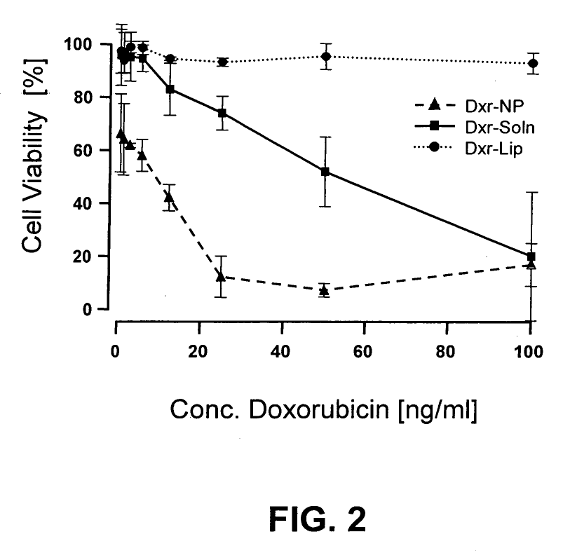

To examine whether a resistance could be overcome, the preparations containing doxorubicin were also tested on doxorubicin-resistant neuroblastoma cells. In these tests, it was found that there was a considerable difference between the various preparations (FIG. 2). The highest cytotoxicity was observed with the nanoparticulate Dxr preparation, which had an IC50 of 14.4 ng/ml. The Dxr solution had a considerably weaker influence on the cell viability. With that solution, the IC50 rose to 53.46 ng/ml, compared to the test with the parenteral UKF-NB3 cells. The liposomal Dxr preparation had no influence on the growth of the UKF-NB3 Dexr-R. cells. Even concentrations of 100 ng/ml of doxorubicin showed no cytotoxic effect.

TABLE 2
IC50 values of Dxr-NP, Dxr-Soln, Dxr liposomes,
in parenteral and resistant UKF-NB3 cells

| | |
|---|---|
| UKF-NB3 Par. | UKF-NB3 Dxr-R. |

| | | | | |
|---|---|---|---|---|
| Dxr-NP | 2.4 | ng/ml | 14.4 | ng/ml |
| Dxr-Soln | 1.6 | ng/ml | 53.5 | ng/ml |
| Dxr-Liposomes | 25.8 | ng/ml | >100.0 | ng/ml |

The results of the cytotoxicity test clearly show that doxorubicin, in different preparations, strongly inhibits the cell growth of tumour cells. In non-resistant cells, the Dxr nanoparticles and the Dxr solution showed a comparable effect. However, if the formation of resistance occurs during a therapy with cytostatic agents, the nanoparticulate Dxr preparation is superior to an active agent solution. Liposomal Dxr preparations, on the other hand, are not capable of overcoming resistance mechanisms of tumour cells.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. Use of nanoparticles comprising a matrix of at least one protein in which at least one antineoplastic active agent is embedded, for manufacturing a medicament for treating tumours which are resistant to chemotherapeutic agents.

2. The use of nanoparticles comprising a matrix of at least one protein in which at least one antineoplastic active agent is embedded, for treating tumours which are resistant to chemotherapeutic agents.

3. The use according to claim 1, wherein said protein is selected from the group consisting of albumin, gelatine, casein and immunoglobulins.

4. The use according to claim 1, wherein said antineoplastic active agent is selected from the group consisting of the cytostatic agents.

5. The use according to claim 1, wherein said antineoplastic active agent is selected from the group consisting of mistletoe preparations, vinblastine, vincristine, vindesine, vinorelbine, etoposide, teniposide, nimustine, carmustine, lomustine, cyclophosphamide, estramustine, melphalan, ifosfamide, trofosfamide, chlorambucil, bendamustine, dacarbazine, busulfan, procarbazine, treosulfan, temozolomide, thiotepa, daunorubicin, doxorubicin, epirubicin, mitoxantrone, idarubicin, bleomycin, mitomycin, Dactinomycin, methotrexate, fludarabine, cladribine, mercaptopurine, thioguanine, cytarabine, gemcitabine, fluorouracil, capecitabine, paclitaxel, docetaxel, carboplatin, cisplatin and oxaliplatin.

6. The use according to claim 1, wherein said antineoplastic active agent is selected from the group consisting of platinum compounds, amsacrine, irinotecan, hydroxycarbamide, pentostatin, porfimer sodium, aldesleukin, tretinoin and asparaginase.

7. The use according to claim 1, wherein the surface of the nanoparticles comprises polyethylene glycol molecules or drug targeting ligands.

8. The use according to claim 7, wherein said polyethylene glycol molecules are monofunctional or bifunctional polyethylene glycol derivatives.

9. The use according to claim 7, wherein said drug targeting ligands are selected from the group consisting of trastuzumab, cetuximab, antibodies recognising tumour-specific proteins, transferrin and galactose.

10. The use according to claim 1, wherein the nanoparticles have a size of 100 to 600 nm.

11. The use according to claim 3, wherein said protein is human serum albumin.

12. The use according to claim 4, wherein said cytostatic agents are selected from the group consisting of plant cytostatic agents and chemically defined cytostatic agents.

13. The use according to claim 12, wherein said chemically defined cytostatic agents are selected from the group consisting of the alkaloids, the podophyllotoxins, podophyllotoxin derivatives, alkylating agents, the cytotoxic antibiotics and the antimetabolites.

14. The use according to claim 13, wherein said alkaloids are selected from the group consisting of the vinca-alkaloids, wherein said alkylating agents are selected from the group consisting of nitrosoureas and nitrogen mustard analogues, wherein said cytotoxic antibiotics are selected from the group consisting of the anthracyclines and wherein said antimetabolites are selected from the group consisting of the folic acid analogues, purine analogues and pyrimidine analogues.

15. The use according to claim 10, wherein the nanoparticles have a size of 100 to 400 nm.

16. The use according to claim 15, wherein the nanoparticles have a size of 100 to 200 nm.

17. The use according to claim 2, wherein said protein is selected from the group consisting of albumin, gelatine, casein and immunoglobulins.

18. The use according to claim 17, wherein said protein is human serum albumin.

19. The use according to claim 2, wherein said antineoplastic active agent is selected from the group consisting of the cytostatic agents.

20. The use according to claim 19, wherein said cytostatic agents are selected from the group consisting of plant cytostatic agents and chemically defined cytostatic agents.

21. The use according to claim 20, wherein said chemically defined cytostatic agents are selected from the group consisting of the alkaloids, the podophyllotoxins, podophyllotoxin derivatives, alkylating agents, the cytotoxic antibiotics and the antimetabolites.

22. The use according to claim 21, wherein said alkaloids are selected from the group consisting of the vinca-alkaloids, wherein said alkylating agents are selected from the group consisting of nitrosoureas and nitrogen mustard analogues, wherein said cytotoxic antibiotics are selected from the group consisting of the anthracyclines and wherein said antimetabolites are selected from the group consisting of the folic acid analogues, purine analogues and pyrimidine analogues.

23. The use according to claim 2, wherein said antineoplastic active agent is selected from the group consisting of mistletoe preparations, vinblastine, vincristine, vindesine, vinorelbine, etoposide, teniposide, nimustine, carmustine, lomustine, cyclophosphamide, estramustine, melphalan, ifosfamide, trofosfamide, chlorambucil, bendamustine, dacarbazine, busulfan, procarbazine, treosulfan, temozolomide, thiotepa, daunorubicin, doxorubicin, epirubicin, mitoxantrone, idarubicin, bleomycin, mitomycin, Dactinomycin, methotrexate, fludarabine, cladribine, mercaptopurine, thioguanine, cytarabine, gemcitabine, fluorouracil, capecitabine, paclitaxel, docetaxel, carboplatin, cisplatin and oxaliplatin.

24. The use according to claim 2, wherein said antineoplastic active agent is selected from the group consisting of platinum compounds, amsacrine, irinotecan, hydroxycarbamide, pentostatin, porfimer sodium, aldesleukin, tretinoin and asparaginase.

25. The use according to claim 2, wherein the surface of the nanoparticles comprises polyethylene glycol molecules or drug targeting ligands.

26. The use according to claim 25, wherein said polyethylene glycol molecules are monofunctional or bifunctional polyethylene glycol derivatives.

27. The use according to claim 25, wherein said drug targeting ligands are selected from the group consisting of trastuzumab, cetuximab, antibodies recognising tumour-specific proteins, transferrin and galactose.

28. The use according to claim 2, wherein the nanoparticles have a size of 100 to 600 nm.

29. The use according to claim 28, wherein the nanoparticles have a size of 100 to 400 nm.

30. The use according to claim 29, wherein the nanoparticles have a size of 100 to 200 nm.

31. A method of producing nanoparticles for treating resistant tumour cells, comprising the steps of:
dissolving at least one protein and at least one active agent in an aqueous medium;
precipitating said at least one protein in the form of nanoparticles by controllably adding a non-solvent for said at least one protein to form precipitated nanoparticles;
stabilising the precipitated nanoparticles by adding a crosslinking agent or by heat treatment; and
purifying the nanoparticles by washing or centrifuging.

32. The method according to claim 31 wherein said solvent is an organic solvent.

33. The method according to claim 32 wherein said organic solvent is ethanol.

34. The method according to claim 31, wherein the molar ratio of active agent to protein is 5:1 to 50:1.

35. The method according to claim 31, wherein said crosslinking agent is selected from the group of substances consisting of glutaraldehyde, formaldehyde, bifunctional succinimides, isothiocyanates, sulfonyl chlorides, maleimides and Pyridyl disulfides.

36. The method according to claim 31, wherein said heat treatment step is carried out at 80° C. for 1 hour, or at 70° C. for 2 hours.

37. The method according to claim 31, further comprising the step of modifying the surface of the nanoparticles by covalent binding of at least one of PEG derivatives and drug targeting ligands.

38. The method according to claim 37, wherein said drug targeting ligand is selected from the group consisting of antibodies which recognise tumour-specific proteins, trastuzumab, cetuximab, transferrin and galactose.

39. Nanoparticles for treating resistant tumour cells, said nanoparticles comprising a matrix of at least one protein and at least one active agent embedded in said matrix, wherein said at least one protein is selected from the group consisting of albumin, gelatine, casein and immunoglobulins, and wherein the surface of said nanoparticles comprises a substance selected from the group consisting of polyethylene glycol molecules and drug targeting ligands.

40. The nanoparticles according to claim 39, wherein the active agent is an antineoplastic active agent.

41. The nanoparticles according to claim 39, wherein said at least one protein is human serum albumin.

41. The nanoparticles according to claim 39, wherein the active agent is selected from the group consisting of the cytostatic agents, comprising plant cytostatic agents, chemically defined cytostatic agents from the groups of the alkaloids, especially the vinca-alkaloids, the podophyllotoxins, podophyllotoxin derivatives, alkylating agents, especially nitrosoureas, nitrogen mustard analogues, the cytotoxic antibiotics, preferably the anthracyclines, the antimetabolites, especially the folic acid analogues, purine analogues and pyrimidine analogues.

42. The nanoparticles according to claim 39, wherein the active agent is selected from the group consisting of mistletoe preparations, vinblastine, vincristine, vindesine, vinorelbine, etoposide, teniposide, nimustine, carmustine, lomustine, cyclophosphamide, estramustine, melphalan, ifosfamide, trofosfamide, chlorambucil, bendamustine, dacarbazine, busulfan, procarbazine, treosulfan, temozolomide, thiotepa, daunorubicin, doxorubicin, epirubicin, mitoxantrone, idarubicin, bleomycin, mitomycin, Dactinomycin, methotrexate, fludarabine, cladribine, mercaptopurine, thioguanine, cytarabine, gemcitabine, fluorouracil, capecitabine, paclitaxel, docetaxel, carboplatin, cisplatin and oxaliplatin.

43. The nanoparticles according to claim 40, wherein said antineoplastic active agent is selected from the group consisting of platinum compounds, amsacrine, irinotecan, hydroxycarbamide, pentostatin, porfimer sodium, aldesleukin, tretinoin and asparaginase.

44. The nanoparticles according to claim 39, wherein said polyethylene glycol molecules are monofunctional or bifunctional polyethylene glycol derivatives.

45. The nanoparticles according to claim 39, wherein said drug targeting ligands are selected from the group consisting of trastuzumab, cetuximab, antibodies recognising tumour-specific proteins, transferrin and galactose.

46. The nanoparticles according to claim 39, wherein said nanoparticles have a size in the range of 100 to 600 nm

47. The nanoparticles according to claim 46, wherein said nanoparticles have a size in the range of 100 to 400 nm.

48. The nanoparticles according to claim 47, wherein said nanoparticles have a size in the range of 100 to 200 nm.
